(19) United States
(12) Patent Application Publication
(10) Pub. No.: US 2025/0298101 A1
(43) Pub. Date: Sep. 25, 2025

(54) MAGNETIC RESONANCE-BASED CATHETER LOCALIZATION

(71) Applicant: CASE WESTERN RESERVE UNIVERSITY, Cleveland, OH (US)

(72) Inventors: Ridaa Zahra Ali, Willoughby, OH (US); Rasim Boyacioglu, Cleveland Heights, OH (US); M. Cenk Cavusoglu, Beachwood, OH (US); Andrew Dupuis, Cleveland Heights, OH (US); Mark Griswold, Shaker Heights, OH (US); Daniel Herzka, Solon, OH (US)

(21) Appl. No.: 19/084,469

(22) Filed: Mar. 19, 2025

Related U.S. Application Data
(60) Provisional application No. 63/567,155, filed on Mar. 19, 2024.

(51) Int. Cl. G01R 33/28; A61B 34/20; A61M 25/01; G01R 33/54; G01R 33/56

(52) U.S. Cl. CPC G01R 33/285; A61B 34/20; G01R 33/543; G01R 33/5608; A61B 2034/2051; A61B 2034/2065; A61M 25/0127

(57) ABSTRACT

In an example, a method includes controlling electrical current to an actuation coil disposed about an elongate flexible body of a device within a field of view of magnetic resonance (MR) imaging system. The MR imaging system can be controlled to provide radio frequency pulses at one or more off-resonant frequencies to excite off-resonance spins near the actuation coil, such that the acquired MR data is representative of the off-resonance excitation. MR data can be provided based on MR signals acquired by the MR imaging system, in which at least some of the MR data includes the MR signals with the device within the field of view. Images can be reconstructed based on the MR data to provide reconstructed image data and localization data can be provided representative of at least one of a location, orientation and/or shape of the device based on the reconstructed image data.

### CROSS-REFERENCE TO RELATED APPLICATIONS

This application claims the benefit of priority to U.S. Provisional App. No. 63/567, 155, filed Mar. 19, 2024, which is incorporated herein by reference in its entirety.

### GOVERNMENT FUNDING

This invention was made with government support under HL153034 and HL163991 awarded by the National Institutes of Health; and 1563805 and 1563805 awarded by the National Science Foundation. The government has certain rights in the invention.

### TECHNICAL FIELD

This description relates to magnetic resonance-based catheter localization.

### BACKGROUND

Magnetic resonance (MR)-based catheter tracking is of interest because MR- guided cardiovascular interventions offer surgical-level exposure in a minimally invasive manner while decreasing (or eliminating) exposure to radiation and nephrotoxic radiocontrast and need for x-ray protective lead aprons which can cause musculoskeletal injury. Catheter tracking approaches generally can be divided into active and passive approaches which differ in that the former involves incorporating an active element to the catheter.

### SUMMARY

This description relates to MRI-based catheter localization, and includes multiple approaches, which can be used separately or in some combination, to localize a catheter using magnetic resonance (MR) imaging.

In one example, a system includes one or more non-transitory media and a processor. The non-transitory media can store data and executable instructions. The processor is configured to access the non-transitory media and the execute instructions. Image reconstruction code is programmed to reconstruct images based on acquired magnetic resonance (MR) data and provide reconstructed image data, in which at least some of the MR data includes a representation of a device within a field of view. Localization code programmed to provide localization data representative of at least one of a location, orientation and/or shape of the device based on the reconstructed image data. Off-resonance control code programmed to control an MR imaging system to provide radio frequency (RF) pulses at one or more off-resonant frequencies to excite off-resonance spins near a current- carrying coil carried by the device within the field of view. The acquired MR data is representative of the off-resonance excitation during MR image acquisition.

In another example, a method includes controlling electrical current to at least one actuation coil disposed about an elongate flexible body of a device within a field of view of magnetic resonance (MR) imaging system. The method also includes controlling the MR imaging system to provide radio frequency (RF) pulses at one or more off-resonant frequencies to excite off-resonance spins near the at least one actuation coil within the field of view, such that the acquired MR data is representative of the off-resonance excitation during MR image acquisition. The method also includes providing MR data based on MR signals acquired by the MR imaging system, in which at least some of the MR data includes the MR signals with the device within the field of view. The method also includes reconstructing images based on the MR data to provide reconstructed image data. The method also includes providing localization data representative of at least one of a location, orientation and/or shape of the device based on the reconstructed image data.

In another example, a non-transitory machine-readable medium can store executable instructions, in which the instructions are operative to cause a processor to perform the foregoing method.

### BRIEF DESCRIPTION OF THE DRAWINGS

FIG. 1 depicts an example of a system environment, showing an example localization method.

FIG. 2 is a flow diagram depicting an example method of localizing a catheter using off-resonance excitation during MR acquisition.

FIG. 3 is a flow diagram depicting an example method of localizing a catheter using rephaser control during MR acquisition.

FIG. 4 is an image of an active coil that is acquired using the rephaser control of FIG. 3 as part of the MR imaging sequence.

FIG. 5 is a flow diagram depicting an example method of localizing a catheter using reverse polarization reconstruction as part of the MR imaging sequence.

FIG. 6 is an MR image reconstructed using forward polarization.

FIG. 7 is an MR image reconstructed using reverse polarization based on the same MR image data as FIG. 6.

FIG. 8 is a flow diagram depicting an example method of localizing a catheter based on analysis of an acquired MR image relative to an expected MR signal.

FIGS. 9 and 10 illustrate examples of locations determined according to different methods and across a number of images for the respective methods.

FIG. 11 depicts an example of an intervention system that can implement a localization method.

### DETAILED DESCRIPTION

This description relates to MRI-based catheter localization, and includes multiple approaches, which can be used separately or in various combinations, to localize a device (e.g., a catheter) using magnetic resonance (MR) imaging. A first example approach is device localization using off-resonance excitation (during MR acquisition). A second example approach is catheter localization using bright marker refocusing (during MR acquisition). A third example approach is localizing a device using reverse polarization reconstruction (during MR data reconstruction/processing). A fourth example approach is pattern matching based on acquired images or one-dimensional projections thereof with computed signal patterns (e.g., simulated Biot-Savart signal patterns) to localize a device. As described herein, any or all of the first through fourth examples can be combined for localizing a device, which can be a catheter.

As a further example, the systems and methods described herein further can be to localize a robotic ablation catheter using the off-resonance inherent to spins near a current- carrying coil that is carried by the catheter. By selectively exciting these off-resonant spins, signal near the catheter can be magnified while signal from other parts of the imaging volume is suppressed. This data can be reconstructed multiple times with variations (using images, weight matrices, and coil sensitivity maps the ‘normal way’ or using the conjugates of these quantities) that produce slightly different images. Then, these reconstructions are fit to Gaussian distributions, compared to Biot-Savart predictions, and analyzed for location of increased (e.g., maximum) signal. By combining these reconstruction and analysis methods, several estimates for catheter coil location are generated which are then combined using goodness of fit or other optimization metrics to generate stable and accurate catheter localization.

Catheter interventions are common and have multiple applications in medical practice; thus, catheter localization and tracking represent an important challenge to be addressed. MR-based catheter tracking is of interest because MR-guided cardiovascular interventions offer surgical-level exposure in a minimally invasive manner while decreasing (or even completely eliminating) exposure to radiation and nephrotoxic radiocontrast and need for x-ray protective lead aprons which can cause musculoskeletal injury. The example embodiments described herein address challenges associated with catheter tracking by localizing an ablation catheter without requiring contrast agent, or modification to or addition of coils or fiducials to the ablation catheter. In examples used to localized a catheter, the catheter can be an active catheter. In this context, the term active can mean that the catheter is actuated to move (e.g., axial or transverse motion), but active can also be also refer to activation of coils as part of a localization scheme, which can involve the ability to turn catheter currents on and off for localization.

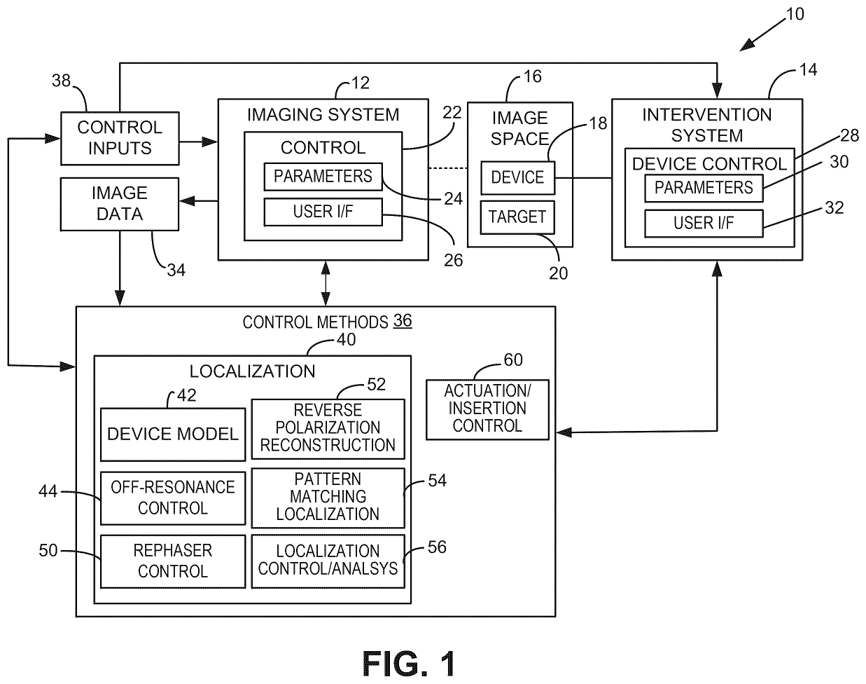

FIG. 1 depicts an example system environment 10, which can be configured to implement systems and methods described herein. The system environment 10 includes an imaging system 12 and an intervention system 14. The imaging system 12 can be external to the patient's body or internal to the patient's body and configured for intra-operative imaging of an image space 16. As used herein, intra-operative refers to utilizing the imaging during a procedure, such as a diagnostic procedure and/or an interventional procedure that is being performed with respect to the patient's body, which forms at least a portion of the image space 16. The image space 16 includes at least a portion of a patient's body and thus includes a device (e.g., an instrument moveable within the patient's body) 18 and a target site (e.g., an anatomical target) 20. In examples described herein, the device 18 is an active catheter (e.g., an ablation catheter), but the systems and methods described herein can be used for localization of other types of devices. The target 20 is a desired location in the image space 16 to which the device 18 is being moved. The target 20, which can be fixed or moveable, further may be an intermediate point along a trajectory or a final destination on such trajectory.

By way of example, the imaging system 12 can be an intra-operative MR imaging modality. Other slice based intra-operative imaging modalities and/or projection based intra-operative imaging modalities can be used in other examples. The imaging system 12 provides image data 34 that corresponds to a time-based sequence of images of the image space 16, including the target 20 and the device located therein. Changes in the sequence of images provided by the image data 34, for example, can represent movement of the target 20, the device 18 as well as other matter (e.g., surrounding tissue) measurable by the imaging modality employed by the imaging system 12. The imaging data can be provided to a display to visualize the sequence of images acquired over time as well as be stored in memory (e.g., one or more machine readable storage media) for image analysis. The imaging system 12 can include image processing techniques to characterize tissue and other objects (e.g., including device 18 and target 20) in the image space 16, such that image data 34 can include processed images. For a given time step, the image data 34 can be a two-dimensional spatial visualization (e.g., an image across a plane) or three-dimensional spatial visualization (e.g. an image across a volume), and thus the image data can include pixels or voxels, accordingly. The analysis and processing of the images disclosed herein thus is with respect to the pixels or voxels in the image data 34.

The imaging system 12 includes a control system 22 to control imaging the image space 16 over time (e.g., over one or more imaging time steps). The control system 22 can control the imaging process over time according to a set of time variable input parameters 24. One or more of the input parameters 24, including activation and deactivation of the imaging system 12, can be provided in response to a user input via a user interface 26. The user interface 26 can be a human-machine interface to interact with the imaging system 12, such as to set one or more parameters 24 or otherwise configure the imaging system before or during an imaging procedure.

As mentioned, the intervention system 14 includes a device control systems 28 to control the intervention system, which can be employed to implement a medical intervention with respect to a portion of the patient's body located in the imaging space 16. The device control system 28 can control the intervention system 14, including controlling motion and/or positioning of the device 18, over time according to a set of associated operating parameters 30. The device control system 28 also includes a user interface 32 that can be utilized to provide or adjust one or more of the parameters 30 in response to a user input, including activation and deactivation of the intervention system 14. The user interface 32 can include a human-machine interface to interact with the intervention system 14, such as to set one or more of the parameters 30 or otherwise configure and/or control the intervention system 14 before and/or during an interventional procedure.

As a further example, the intervention system 14 is configured to perform a percutaneous intervention with respect to the patient's body. For example, the device 18 is a catheter configured to be inserted percutaneously into the patient's body and advanced to the target 20 for performing a respective procedure (e.g., diagnostic and/or treatment of tissue, such as tissue ablation). The catheter 18 has an arrangement of one or more coils for magnetic actuation disposed along the distal end portion thereof configured to deflect the distal end portion responsive to applied current and/or a magnetic field. In addition to deflection of the distal end portion, the catheter can also be advanced and retracted inside the patient's body from its distal end. One or more of the coils can be multi-axis coils. In an example, the catheter device 18 includes multiple sets of coils at respective spaced apart locations along a distal end portion of a body of the catheter device, such as one set adjacent the distal tip of the catheter and at least another set of coils spaced a distance axially apart from the catheter tip. Other arrangements of coils can be provided on the catheter body.

Examples of coil configurations and control circuitry that can be implemented for the catheter device 18 are disclosed in International Patent Pub. No. WO2024/0196999 (corresponding to PCT App. PCT/US2024/020664, filed Mar. 20, 2024), which is incorporated herein by reference in its entirety. Other catheter and coil configurations can be used in other examples.

In an example, the control system 28 is configured to generate output signals to control the coil in the device 18 such as input electric current selectively provided to energize respective coils as to cause movement of the catheter toward a desired point on a given trajectory between the end point and the target site. The control system 28 can additionally generate output signals to control the axial insertion and retraction of the catheter. For example, the control system 28 includes actuation controls configured to implement the control methods programmed to control device positioning and actuation, such as including inverse kinematics and Jacobian controls to adjust the position of the catheter (or other instrument) toward the target 20 based on active control inputs. In response to the current applied by the intervention system 14, for example, magnetic moments can be generated on each respective coil including the tip or other location where the coils reside along the elongated body portion of the catheter. The magnetic moments generated at each coil sets interact with the magnetic field of the MR scanner implemented at the imaging system 12 to deflect the flexible elongated body of the catheter in a desired direction with respect to a central longitudinal axis thereof.

The catheter 18 also includes a lumen into which one or more tools can be inserted through (e.g., partially or completely through). In some examples, the tool is a stiffening clement (e.g., rigid shaft) that is axially movable along the length of the catheter body. The stiffening element extends longitudinally between proximal and distal ends thereof, and can be straight or have one or more curved portions along its length. In one example, the distal end of the stiffening element can be inserted into the catheter lumen to terminate at a distal insertion point, such as within the flexible distal end portion of the catheter. In another example, the stiffening element can be inserted over and along the length of the catheter body to terminate at an axial position, such as circumscribing part of the distal end portion of the catheter. The presence of the stiffening element within the catheter lumen or along an exterior of the catheter body is configured to inhibit (e.g., prevent) bending of proximal portion of the catheter and control the amount of deflection that can be implemented by the distal portion of the catheter, such as described herein. The position of the flexible tool within the catheter can be fixed (e.g., by a locking mechanism). As an alternative, the elongated tool can be a flexible tool, such as a second flexible catheter, an injection needle, a puncture needle, guidewire, or other elongate flexible tool dimensioned and configured to be inserted within the catheter lumen according to needs of the respective procedure.

The systems and methods described herein thus can be configured to perform motion control of the catheter and the tool, which resides within the catheter according to the motion control of the catheter. As described herein, the motion control of the catheter and the flexible tool therein can include magnetic actuation of the flexible catheter tip and tool within the tip as well as insertion control thereof. In some examples, the intervention system 14 includes robotic actuation tools or instruments, which are inserted into the patient and guided to the target (e.g., a fixed or moving target) 20 based on localization via intra-operative medical imaging (e.g., via imaging system 12). As one example, the intervention system 14 can be implemented as a robotically controlled catheter ablation system in which the catheter tip (e.g., device 18) is to ablate at the target site 20 based on control parameters 30 that vary with respect to time.

In the example of FIG. 1, the control inputs 38 can provide active control for the imaging system 12 and/or the intervention system 14. The control inputs 38 set one or more of the image parameters 24 of the imaging system 12 determined to maximize information gain about the current state of the system 10. Additionally or alternatively, the control inputs 38 also can set one or more of the control parameters 30 of the intervention system 14 also to maximize information gain for the system 10. The control parameters 30 set via the control inputs 38 can be employed to steer and adjust the position of the device (e.g., catheter or needle) in substantially real time. The control inputs 38 to the intervention system 14 thus can be utilized to navigate a tip of the device to the target site 20 in the patient's body as well as to implement relative motion control (e.g., motion cancellation) once at the target site so that the device 18 moves commensurately with movement of the tissue target (e.g., to maintain continuous contact).

As disclosed herein, the system 10 includes one or more control methods 36, which include one or more localization methods 40 and actuation/insertion control methods 60. The more control methods 36, including the localization and actuation/insertion methods 40 and 60, can be implemented as machine-readable instructions (e.g., program code) stored in one or more non-transitory media, which are executable by one or more processors to perform the functions described herein. That is, the control methods 36 can be implemented by a computer device, which has interfaces coupled to the control inputs 38, the source of the image data 34 and the intervention system 14. While the control methods 36 are shown outside of the intervention system 14 and imaging system 12, it is to be understood that the control methods, in whole or in any part thereof, can be implemented within the imaging system 12, the intervention system 14 or in both the imaging and intervention systems.

In the example of FIG. 1, control methods 36 include localization methods 40 and actuation and insertion control methods 60. The actuation and insertion control methods 60 are configured to implemented coordinated control of insertion motion and magnetic actuation (e.g., navigation) of the device 18. For example, the device 18 can be a catheter or other device having a flexible (e.g., bendable or pliant) tip having one or more coils, each of which coils having one or multiple axes. Multi-axis coils help avoid magnetic actuation singularities. Further examples of the actuation and insertion control methods 60 are described in the above-incorporated International Patent Pub. No. WO2024/0196999.

The localization methods 40 can be implemented to control image acquisition to provide the image data 34 and/or control image reconstruction based on acquired MR data 34 to facilitate accurately localizing the device 18. In one example, the localization methods 40 includes off-resonance control 44 (e.g., instructions code executable by a processor) programmed to localize the device 18 based on off-resonance excitations in acquired MR data 34. The off-resonance control 44 can provide commands (e.g., instructions) to the imaging system 12 during MR acquisition, to provide off-resonant radio frequency (RF) pulses that selectively excite off-resonant spins. The off-resonant spins result in signal near the device (e.g., catheter) 18 being magnified while signal from other parts of the imaging space 16 (e.g., anatomy) is suppressed. The off-resonant spins refer to spins that are off- resonant due to their proximity to one or more current-carrying steering coils on the device 18. In examples, the off-resonant RF pulses can be provided at a particular frequency (e.g., frequency range) adapted to achieve off-resonant spins proximal respective coils on the device 18. The frequency can be computed, determined empirically through testing or otherwise determined a priori for a given device. The off-resonance control 44 thus can control the MR scanner to excite off-resonant spins by controlling the magnetic field of the imaging system 12, such as during a localization phase of image acquisition. The off- resonance spins can be identified in each dimension of a multi-dimensional (e.g., 1D, 2D or 3D) coordinate system over a number of acquired images. In some examples, the MR data acquisition described herein can be implemented as set forth in Haase, A., Frahm, J., Matthaci, D., Hanicke, W., & Merboldt, K. D. (1986). FLASH imaging. Rapid NMR imaging using low flip-angle pulses; Journal of Magnetic Resonance (1969), 67(2), 258-266.

Additionally, or as an alternative example, the localization methods 40 includes rephaser control code (e.g., instructions executable by a processor) 50 programmed to localize the device 18 based on bright marker refocusing in acquired MR data 34. The rephaser control can provide commands (e.g., instructions) to the imaging system 12 to remove refocusing (e.g., usually provided by RF refocusing pulses) during MR image acquisition, such as by deactivating (e.g., turning off) a slice rephaser of the imaging system 12 to remove RF refocusing pulses at the end of a slice select gradient. Because the slice rephaser is deactivated in this way, refocusing that would normally occur due to the rephaser gradient is effectively removed. As a result of refocusing being removed, the background features are suppressed and increased brightness is associated with areas proximal to the active coils carried by the device (e.g., catheter) 18. Background suppression is likely due to intravoxel dephasing, and bright areas are associated with the catheter, which acts as a rephasing gradient in its vicinity. The localization methods 40 can thus identify locations of the bright areas in each dimension of a multi-dimensional (e.g., 1D, 2D or 3D) coordinate system based on analysis of a number of acquired images, and provide location data specifying the location (e.g., spatial coordinates) of the device 18.

Additionally, or as an alternative example, the localization methods 40 includes reverse polarization reconstruction code (e.g., instructions executable by a processor) 52 programmed to localize the device 18 based on reverse polarization reconstruction applied to the acquired MR data 34. The reverse polarization reconstruction 52 can be applied to the acquired MR data 34 acquired based on the off-resonance control 44 and/or rephaser control 50 implemented during image acquisition. Alternatively, the reverse polarization code can be applied to image data in the absence of off-resonance excitation or with refocusing by enabling slice rephasing (e.g., without activation of slice rephaser control 50). The reverse polarization reconstruction code 52 can implement a reverse polarization mode during reconstruction to separate the catheter signal from the anatomical signal. The image reconstruction code can apply the reverse polarization to the entire acquired MR data 34. Also, or as an alternative, the image reconstruction code can include related operations such as performing complex conjugations of signals, of weight matrices, and/or of other parameters of the image data or derived therefrom having real and imaginary components. Because the anatomy is effectively suppressed in reverse polarization reconstruction, the reconstructed image includes the device 18, particularly areas near coils that are sensitive to the effects of the coils during image acquisition. The localization methods 40 can thus identify the features (e.g., active coils) in each dimension of a multi-dimensional (e.g., 1D, 2D or 3D) coordinate system over a number of reconstructed images based on the reverse polarization reconstruction 52, and provide corresponding to the location (e.g., coordinates of the device).

In some examples, the image reconstruction described herein (with or without reverse polarization reconstruction) can be implemented according to one of the approaches described in Walsh, D. O., Gmitro, A. F., & Marcellin, M. W. (2000); Adaptive reconstruction of phased array MR imagery. Magnetic Resonance in Medicine: An Official Journal of the International Society for Magnetic Resonance in Medicine, 43(5), 682-690; or in Griswold, M. A., Walsh, D., Heidemann, R. M., Haase, A., & Jakob, P. M. (2002).

Additionally, or as an alternative example, the localization methods 40 includes pattern matching localization code (e.g., executable by a processor) 54 programmed to localize the device 18 based on pattern matching to expected or known patterns. For example, the pattern matching localization code 54 can be applied during image reconstruction to compare the acquired one-dimensional data (either acquired one- dimensional projections or one-dimensional projections generated from acquired two- dimensional images) to expected signal from Biot-Savart simulations, which can be stored in memory as expected signal data. The expected signal data thus can represent the (one- dimensional projection of the) expected magnetic field due to the current carrying coils on the active catheter, which calculation considers the coil set geometry, position, orientation, and current values. The dot product of the expected signal and one-dimensional data is computed for each possible offset of the expected signal to the one-dimensional data to find the point where the value of the dot product is maximal. The pattern matching localization code 54 can provide a location of the device 18 in each dimension of a multi-dimensional (e.g., 1D, 2D or 3D) coordinate system over a number of reconstructed images based, which can be combined to provide the location (e.g., spatial coordinates of the device).

As described herein, the instructions executable by the processor can be programmed to localize the catheter (e.g., determine spatial coordinates) in a respective spatial domain in combination with any one or more of the off-resonance control code 44, the rephaser control code 50, and/or reverse polarization reconstruction code 52. Such other localization method(s) further can be in addition to or as an alternative to the pattern matching localization code 54. One example method is to use one or more peaks of acquired projections; this also can (but does not have to) be used with the other Approaches or combination of the other Approaches described. One method would be to define the catheter coordinate as the location of the global peak in the acquired projection. Another approach would be to constrain the field of interest around recent past or expected locations of the catheter, and then define the catheter coordinate as the location of the global peak in this truncated projection; that is, the projection will be acquired across the entire volume, then will be truncated around the recent past or expected locations of the catheter; then the catheter coordinate will be defined as the location of the global peak in this truncated projection. Furthermore (for any method of determining catheter coordinates—including pattern matching or peak-based approaches), it is possible to acquire more than 3 projections and perform a least squares optimization to find the coordinates. Example of acquiring projections and using peaks to locate an object of interest (e.g., the catheter or other device), which can be implemented by localization methods 40 herein, are described in Dumoulin, C. L., Souza, S. P., & Darrow, R. D. (1993), Real-time position monitoring of invasive devices using magnetic resonance, Magnetic resonance in medicine, 29(3), 411-415.

The localization methods 40 can also include localization control/analysis code (e.g., instructions executable by a processor) 56 programmed to localize the device 18 based on selectively utilizing or combining any one or more of the off-resonance control 44, rephaser control 50, reverse polarization reconstruction 52, and pattern matching localization 54. In some examples, the localization control/analysis code 56 can combine any such acquisition, reconstruction, and/or analysis methods 44, 50, 52, and 54 to generate several different estimates for device coil location. Also, or as an alternative example, the reverse polarization code and/or pattern matching code are implemented as part of the image reconstruction code, or the reverse polarization and/or pattern matching of expected signals are implemented after MR image reconstruction and/or processing. The respective estimates for device coil location can further be analyzed and combined based on determining a goodness of fit metric to determine stable and accurate catheter localization data specifying the catheter location in 3D spatial coordinates. The localization data can be determined in the coordinates of the imaging system (e.g., MR scanner) 12 and transferred to a global coordinate system, a coordinate system of the device 18 or a coordinate system of the patient's body. The ultimate spatial coordinate system can be defined by the user (e.g., in response to a user input instruction) to specify one or more desired spatial domain for the location data describing the position of the catheter. The localization method 40, including one or more of the methods 44, 50, 52, 54, and 56) thus can be executed repeatedly to provide the location data in real time or near real time during MR imaging without requiring contrast agent, or modification to or addition of coils, or adding fiducials to the catheter (e.g., an active ablation catheter).

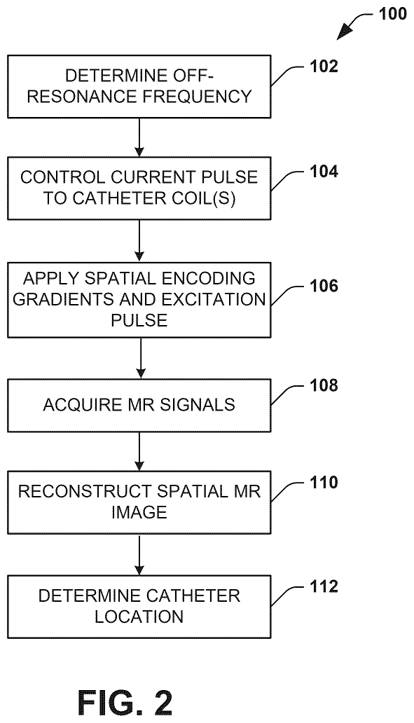

FIGS. 2, 3, 5, and 8 are example methods that can be implemented individually or any combination thereof by the localization method 40. While, for purposes of simplicity of explanation, the methods are shown and described as executing serially, it is to be understood and appreciated that such methods are not limited by the illustrated order, as some aspects could, in other examples, occur in different orders and/or concurrently with other aspects from that disclosed herein. Moreover, not all illustrated features may be required to implement a method. The methods or portions thereof can be implemented as instructions stored in one or more non-transitory machine readable media and be executed by a processor of one or more computing devices, for example. The methods of FIGS. 2, 3, 5, and 8 provide examples of an MR sequence that can be implemented by a system (e.g., the system 10, 500). Accordingly, the methods of FIGS. 2, 3, 5, and 8 can refer to certain aspects of FIGS. 1 and/or 11.

### Off-Resonance Control

FIG. 2 is a flow diagram depicting an example method 100 of localizing a catheter using off-resonance excitation during MR acquisition. The method 100 provides an example of an MR sequence that can be implemented by a system (e.g., the system 10, 500) that includes off-resonance control 44 to localize a catheter (e.g., an active catheter).

At 102, the method includes determining an off-resonance frequency. For example, spins precess at a frequency directly related to the strength of the magnetic field they are exposed to, with the proportionality constant being the gyromagnetic ratio. The precession frequency induced by the main magnetic field of the MRI scanner (e.g., MR imaging system 12) is the Larmor frequency. Thus, the precession frequency of spins exposed to the coil current-induced magnetic field of the active catheter will be offset from Larmor frequency by an amount equal to the gyromagnetic ratio times the vector value of the Bz component of the magnetic field induced by the coil current(s) at locations of these spins. This offset from the Larmor frequency can define the off-resonance frequency determined at 102, which can be used to selectively excite off-resonant spins adjacent to the current- carrying catheter coil as described herein.

At 104, the method includes controlling current pulses to one or more coils of the catheter. For example, actuation/insertion control (e.g., control 60) can control one or more pulse generator circuits (e.g., device control system 28) that are coupled to respective catheter coils. The current pulses that are provided in response to the control at 104 are provided during (e.g., synchronized or gated with) MR acquisition (e.g., excitation), as described herein. One or more of the on-time, off-time, duty cycle, and/or magnitude of the current pulse(s) supplied to the coils of the catheter can be controllable parameters (e.g., set responsive to a user input instruction). The current pulses can be sine waves, square waves or other forms of pulse signals having a duty cycle and magnitude, which can vary depending on the particular coil design. The catheter can be an active catheter, such as including actuation coils and control circuitry described herein (e.g., as disclosed in the above-incorporated International Patent Pub. No. WO2024/0196999).

In a first example, the current control at 104 can be implemented to provide the current pulses as actuation pulses for moving (e.g., insertion and/or steering) the catheter body so the localization can track the catheter while moving. In a second example, the current control at 104 can be implemented to provide the current pulses as localization only pulses having a magnitude that is not sufficient to cause movement (e.g., coil current is below threshold for insertion and/or steering) of the catheter. In a third example, the approaches of the first and second examples can be interleaved to provide current control that includes a combination of both subthreshold and suprathreshold current pulses during MR acquisition.

At 106, the method includes applying spatial encoding gradients and an RF pulse to excite the MR system coils (e.g., of imaging system 12, 508). Unless indicated otherwise, as used herein, the spatial encoding gradients can include one or more of a slice- select gradient, a slice refocusing gradient, a phase-encoding gradient, and a frequency encoding gradient. For example, at 106 a slice-select gradient can be applied along a desired axis (e.g., Z-axis) to spatially localize the excitation to a specific slice. Concurrently with the slice-select gradient, at 106, an RF pulse can be applied at the Larmor frequency corresponding to the desired slice location to excite spins only in the selected slice. In the example of FIG. 2, to implement off-resonance control, current pulses are also applied to catheter coils (e.g., based on the control at 104) and the RF pulse applied at 106 is controlled to be selective to the off-resonance frequency, such as to magnify the signal near the catheter, as described herein. By using an RF pulse at 106 that is selective to the frequency of the spins near the actuating catheter, these spins can be excited based on the electric current in one or more of the conducting actuation coils of the catheter, while the magnetic field decreases with distance from the actuation coils (e.g., according to Biot-Savart Law). Thus, the spins near the catheter coils, which are being activated by current pulses, are exposed to a larger magnitude current-induced magnetic field than spins farther from the catheter. As a result, the excited spins contribute greater to the received MR signal near the catheter's coils than away from the coils (e.g., where a lower MR signal is received).

After applying the RF pulse, the method can also include applying a slice refocusing gradient in the same direction as the slice-select gradient. The slice refocusing gradient can compensate for dephasing caused by the slice-select gradient, ensuring that spins within the selected slice are in phase when signal acquisition begins. This improves slice profile sharpness and minimizes artifacts. In examples where rephaser control is implemented in combination with the off-resonance control of the method 100 (or implemented by rephaser control 50), the slice refocusing gradient would be omitted. The encoding gradients applied at 106 further can include applying a phase-encoding gradient along a perpendicular axis (e.g., Y-axis) to introduce spatially dependent phase shifts, encoding positional information along that axis. The encoding gradients applied at 106 further can include applying a frequency-encoding gradient along the third axis (e.g., X-axis) during signal acquisition to create a linear variation in resonance frequency along this axis, enabling spatial encoding of the signal.

At 108, the method includes acquiring the MR signals from a spatial region (e.g., field of view) in which the catheter is located. For example, the signal acquired at 108 is acquired while the frequency-encoding gradient is active and electrical current is applied concurrently to the catheter coil(s) (e.g., current pulses responsive to the control at 152). The acquired signals represent a combination of spatial frequencies (e.g., k-space data) based on proton spins within the spatial region where the MR field is provided.

At 110, the method includes reconstructing a spatial MR image. For example, the reconstruction can include applying an inverse Fourier transform to the k-space data to reconstruct the spatial image, where each voxel corresponds to a location in 3D space. In examples where off-resonance control is combined with reverse polarization reconstruction (e.g., reconstruction 52, method 200), the reconstruction at 110 can determine reverse polarized components of the MR signals that are acquired at 108. In such example, the reconstruction further can include performing conjugate operations on related parameters, such as conjugations of signals, weight matrices, and/or other. As a result of the off- resonance spins in the method 100, the signals in the image being reconstructed are magnified near the catheter coils (e.g., increased signal magnitude) compared to signals from other parts of the imaging volume away from the catheter coils that are suppressed.

At 112, the method includes determining catheter location (e.g., spatial coordinates) based on the reconstructed MR spatial image (at 110). One example approach to determine location is to use one or more peaks of acquired projections (e.g., 1D projections). For example, the MR data acquired at 108 can be acquired in one-dimension. The MR data can also be acquired (at 108) in 2D (e.g., as images of pixels) or 3D (e.g., as volumes of voxels). Thus, after reconstruction (at 110), 2D images can be summed along either dimension to generate corresponding 1D projections. Similarly, after reconstruction (at 110), 3D volumes can be summed along any of the dimensions to generate 1D projections.

As a further example, one approach to determine the location of the catheter (e.g., spatial coordinates for one or more coils carried by the catheter) is to define the catheter coordinate as the location of the global peak in an acquired projection. Also, or as an alternative, another approach is to constrain the field of interest around recent past or expected locations of the catheter, and then define the catheter coordinate as the location of the global peak in this truncated projection. As a further example, the projection will be acquired across the entire volume, then will be truncated around the recent past or expected locations of the catheter, and the catheter coordinate can be defined as the location of the global peak in this truncated projection. In some examples, the catheter localization at 112 further can implement the pattern matching method described herein (e.g., by pattern matching localization code 54 or method 300). Also, or alternatively (for any method of determining catheter coordinates, including peak-based approaches or the pattern matching method 54, 300), it is possible to acquire more than 3 projections and perform a least squares optimization to ascertain the spatial coordinates of the catheter at 112.

As described herein (sec, e.g., FIGS. 1 and/or 11), the location can be used to control generating an image that includes a graphical representation of the catheter superimposed at an image location based on the location determined at 112. Also, or alternatively, the actuation and/or steering of the catheter through application of electrical current to the catheter actuation coils can be controlled based on the location determined at 112. Also, or alternatively, the delivery of ablation energy (e.g., for cryoablation, radio- frequency ablation, irreversible or reversible electroporation) or other forms of treatment provided by a treatment delivery device on the catheter can be controlled based on the location determined at 112.

### Rephaser Control

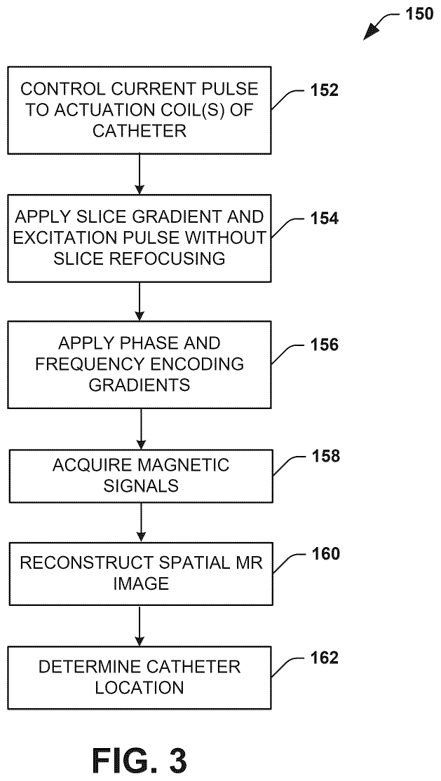

FIG. 3 is a flow diagram depicting an example method 150 of localizing a catheter using rephaser control during MR acquisition. The method 150 provides an example of an MR sequence that can be implemented by a system (e.g., the system 10, 500) that includes bright marker refocusing during MR acquisition to facilitate localization of an active catheter. The method 150 includes an overall workflow that is similar to the method 100 of FIG. 2. Accordingly, further details about certain aspects of the method 150 can be found with reference to corresponding parts of the method 100.

At 152, the method 150 includes controlling current pulses to one or more coils of the catheter (e.g., one or more coils configured to implement insertion and/or steering of a body of the catheter). For example, actuation/insertion control (e.g., control 60) controls one or more pulse generator circuits (e.g., device control system 28) that are coupled to respective catheter coils. The current pulses to one or more catheter coils responsive to the control at 152 are implemented during (e.g., synchronized or gated with) MR acquisition (e.g., excitation) and can be provided for actuation/steering and/or localization as described herein.

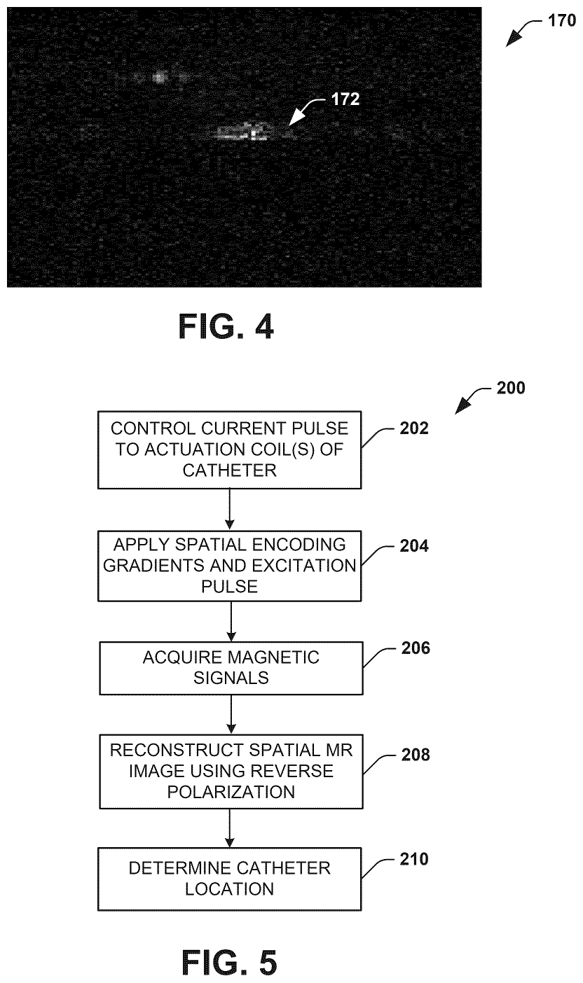

At 154, the method includes applying a slice-select gradient and an RF pulse without including a slice refocusing gradient. Because the slice refocusing gradient is not applied at the end of the slice select gradient, unrefocused spins result in the through-slice direction. The unrefocused spins lead to background suppression likely due to intravoxel dephasing and bright areas associated with the catheter, which acts as a rephasing gradient in its vicinity. Thus, by omitting slice refocusing gradient background/anatomical signal can be suppressed and thereby enhance the MR signal from the catheter coils. By removing this gradient (that is, by not including the slice rephaser gradient in the MR imaging sequence), the background signal is suppressed; however, the areas associated with the catheter still contribute a bright signal (sec, e.g., FIG. 4) because the catheter operates as a rephasing gradient locally in the region adjacent the catheter. Concurrently with the slice-select gradient, at 154, an RF pulse can be applied to the MR system coils to excite spins in the selected slice.

The RF pulse applied at 154 can be applied at the Larmor frequency corresponding to the desired slice location to excite spins only in the selected slice. In an example where the rephaser control is implemented in combination with the off-resonance control, the RF pulse(s) applied at 154 can be controlled to be selective to the off-resonance frequency of the current to the coils to magnify the signal near the catheter along Bz, such as described herein. By using an RF pulse at 154 that is selective to the frequency of the spins near the actuating catheter, the excited spins can contribute greater to the received MR signals near the catheter's coils than away from the coils (e.g., where a lower MR signal is received).

At 156, the method includes applying respective phase and frequency encoding gradients. The phase-encoding gradient can be applied at 156 along a perpendicular axis (e.g., Y-axis) to introduce spatially dependent phase shifts, encoding positional information along that axis. The encoding gradients can be applied at 156 further can include applying a frequency-encoding gradient along the third axis (e.g., X-axis) during signal acquisition to create a linear variation in resonance frequency along this axis, enabling spatial encoding of the signal.

At 158, the method includes acquiring the MR signal within a spatial region (e.g., field of view) containing the catheter. For example, the signal acquired at 158 is acquired while the frequency-encoding gradient (at 156) is active and current is concurrently applied to the catheter coil(s) (e.g., responsive to the control at 152). The acquired signal represents a combination of spatial frequencies (e.g., k-space data) based on proton spins within the spatial region where the MR field is provided.

At 160, the method includes reconstructing a spatial MR image. For example, the reconstruction can include applying an inverse Fourier transform to the k-space data to reconstruct the spatial image, where each voxel corresponds to a location in 3D space. As a result of the omitting slice refocusing gradient at 154 of the method 100, background signals are suppressed in the image being reconstructed, resulting in a relative increase in signal near the catheter coils (e.g., increased signal magnitude). The reconstruction at 160 can be performed using reverse polarization reconstruction, as described herein. In examples where the rephaser control of the method 150 is combined with reverse polarization reconstruction (e.g., reconstruction 52, method 200), the reconstruction at 160 can determine reverse polarized components of the MR signals acquired at 158. In such example, the reconstruction further can include performing conjugate operations on signals and related parameters, such as complex conjugations of imaginary components of one or more of acquired signals, weight matrices, and/or other parameters utilized during reconstruction, such as described herein.

At 162, the method includes determining catheter location based on the reconstructed MR spatial image (at 110). As described herein, the catheter location (e.g., spatial coordinates or one or more catheter coils) can be determined according to any approach(es) described herein, including peak-based approaches and/or pattern matching (e.g., pattern matching method 54, 300).

FIG. 4 is an example of a reconstructed MR image 170 depicting an active coil 172 that is carried by a catheter that was acquired using the rephaser control of FIG. 3 as part of the MR imaging sequence. It can be seen that the rephaser control results in the background signal being suppressed.

### Reverse Polarization Reconstruction

FIG. 5 is a flow diagram depicting an example method 200 of localizing a catheter using reverse polarization reconstruction. The method 200 provides an example of an MR sequence that can be implemented by a system (e.g., the system 10, 500) that includes reverse polarization reconstruction during MR acquisition to facilitate localization of an active catheter. The method 200 includes an overall workflow that is similar to the method 100 of FIG. 2. Accordingly, further information about certain aspects of the method 200 can be found with reference to corresponding parts of the method 100 as well as the system 10.

At 202, the method 200 includes controlling current pulses to one or more coils of the catheter (e.g., one or more coils configured to implement insertion and/or steering of the catheter). For example, actuation/insertion control (e.g., control 60) controls one or more pulse generator circuits (e.g., device control system 28) that are coupled to respective catheter coils. The current pulses to one or more catheter coils responsive to the control at 202 are implemented during (e.g., synchronized or gated with) MR acquisition (e.g., excitation). Also, or as an alternative, the current pulses can be provided for actuation/steering and/or localization, as described herein.

At 204, the method includes applying spatial encoding gradients and an RF pulse to excite the MR system coils (e.g., of imaging system 12, 508). For example, the gradients applied at 204 can include a slice-select gradient applied along a desired axis (e.g., Z-axis) to spatially localize the excitation to a specific slice. Concurrently with the slice- select gradient, at 204, an RF pulse can be applied to the MR system coils. The RF pulse applied at 204 can be applied at the Larmor frequency corresponding to the desired slice location to excite spins only in the selected slice.

In examples where the repolarization reconstruction is implemented in combination with off-resonance control (e.g., control 44, method 100), the RF pulse(s) applied to the MR coils at 204 can be controlled to be selective to the off-resonance frequency of the current applied to the coils to magnify the signal near the catheter along Bz, such as described herein. By using an RF pulse that is selective to the frequency of the spins (offset from the Larmor frequency) near the actuating catheter, the excited spins can contribute greater to the received MR signals near the catheter's coils than away from the coils (e.g., where a lower MR signal is received).

In some examples, the reverse polarization reconstruction of the method 200 can be implemented in combination with the rephaser control (e.g., rephaser control 50, method 150) in which the slice refocusing gradient is omitted from the gradients applied at 204. As a result of the omitting slice refocusing gradient at 204 of the method 200, background signals are suppressed in the image being reconstructed, resulting in a further relative increase in MR signals near the catheter coils (e.g., increased signal magnitude) as represented in the reverse polarization components thereof.

The gradients applied at 204 can also include applying respective phase and frequency encoding gradients. The phase-encoding gradient can be applied at 204 along a perpendicular axis (e.g., Y-axis) to introduce spatially dependent phase shifts, encoding positional information along that axis. The encoding gradients can be applied at 204 further can include applying a frequency-encoding gradient along the third axis (e.g., X-axis) during signal acquisition to create a linear variation in resonance frequency along this axis, enabling spatial encoding of the signal.

At 206, the method includes acquiring the MR signals within a spatial region (e.g., field of view) containing the catheter. For example, the signal acquired at 206 can be acquired while the frequency-encoding gradient (at 204) is active and electrical current pulses are being applied to the catheter coil(s) (e.g., responsive to the control at 202). The acquired signals represent a combination of spatial frequencies (e.g., k-space data) based on proton spins within the spatial region where the MR field is provided. The acquired MR signals can be stored in memory as MR data for further processing as described herein.

At 208, the method includes reconstructing a spatial MR image using reverse polarization. As an example, at 208, the acquired MR signals can be decomposed into separate forward and reverse polarization components, which can separate signal components representative of background/anatomy signal and signal from the coupled coil. An inverse Fourier transform can be applied to the k-space data to reconstruct the spatial image based on reverse polarization components of the acquired MR signals. A corresponding image can be generated (see, e.g., FIG. 7) based on reconstructed MR data derived from the reverse polarization components (without forward polarization components). Thus, in the example method 200, the electrical current used to actuate and/or steer the catheter (e.g., current in the actuation coils as well as the wires leading to these coils) creates the magnetic field with reverse (and forward) polarized components. As a result, the systems and methods implementing reverse polarization reconstruction, can be implemented without including a separate coupled coil fiducial marker on the catheter. Furthermore, coil sensitivity maps can be obtained without device information by acquiring images without the device in the field of view (not obtained using k-space filtering as in existing approaches). In some examples, in addition to reverse polarization reconstruction at 208, the method 300 further can include performing complex conjugate mathematical operations on the MR data prior to or as part of the reconstruction at 208 (e.g., on acquired MR signals). Also, or alternatively, the method 300 can include calculating conjugates of intermediate parameters of image reconstruction, such as by conjugating related reverse polarization parameters, such as conjugations of MR signals, weight matrices, and/or other parameters.

At 210, the method includes determining catheter location based on the reconstructed MR spatial image (at 208). As described herein, the catheter location (e.g., spatial coordinates or one or more catheter coils) can be determined according to any approach(es) described herein, including peak-based approaches and/or pattern matching (e.g., pattern matching method 54, 300).

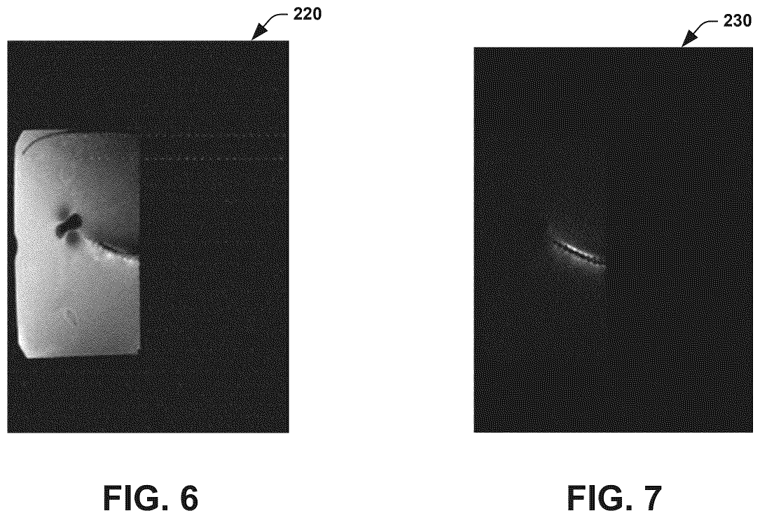

FIG. 6 is an example of an MR image 220 reconstructed based on reconstruction of forward polarization components. FIG. 7 is an example MR image 230 reconstructed using reverse polarization components based on the same MR image data as FIG. 6. A comparison of FIGS. 6 and 7 demonstrates that in the reverse polarized image 230, the background is suppressed and the signal along the catheter wires is bright.

### Pattern Matching

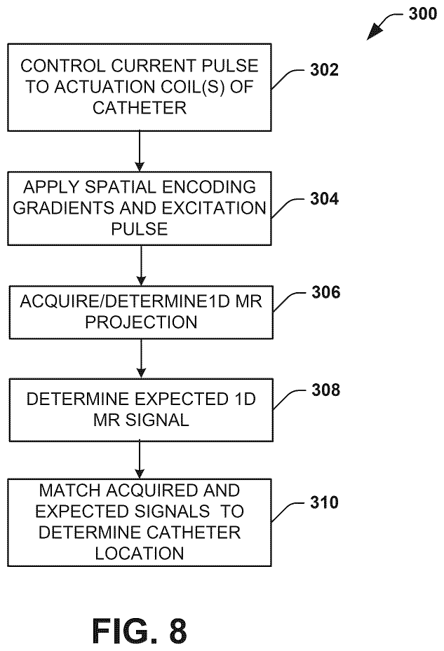

FIG. 8 is a flow diagram depicting an example method 300 of an MR sequence that includes localizing a catheter based on pattern matching. The method 200, which can be implemented by a system (e.g., system 10, 500), includes an overall workflow that is similar to the method 100 of FIG. 2. Accordingly, further information about certain aspects of the method 300 can be found with reference to corresponding parts of the method 100 as well as the system 10. As described herein, localization by pattern matching can include a comparative analysis (e.g., computing a similarity metric) of a reconstructed MR image, or a projection thereof, relative to an expected (e.g., simulated) MR signal along the same dimension(s). Pattern matching provides a robust approach that can be implemented by systems and methods herein for localizing an active catheter and may be utilized separately or in combination with any of the other localization functions described herein.

At 302, the method 300 includes controlling current pulses to one or more coils of the catheter (e.g., one or more coils configured to implement insertion and/or steering of a body of the catheter). For example, actuation/insertion control (e.g., control 60) controls one or more pulse generator circuits (e.g., device control system 28) that are coupled to respective catheter coils. The current pulses to one or more catheter coils responsive to the control at 302 are implemented during (e.g., synchronized or gated with) MR acquisition (e.g., excitation). Also, or as an alternative, the current pulses can be provided for actuation/steering and/or localization of the active catheter, as described herein.

At 304, the method includes applying spatial encoding gradients and an RF pulse to excite the MR system coils (e.g., of imaging system 12, 508). For example, the gradients applied at 304 can include a slice-select gradient applied along a desired axis (e.g., Z-axis) to spatially localize the excitation to a specific slice. Concurrently with the slice-select gradient, at 304, an RF pulse can be applied to the MR system coils. The RF pulse applied at 304 can be applied at the Larmor frequency corresponding to the desired slice location to excite spins only in the selected slice.

In examples where the pattern matching functionality is implemented in combination with off-resonance control (e.g., control 44, method 100), the RF pulse(s) applied to the MR coils at 304 can be controlled to be selective to the off-resonance frequency of the current applied to the coils to magnify the signal near the catheter along Bz, such as described herein. Also, or as an alternative, in some examples, the pattern matching method 300 can be implemented in combination with the rephaser control (e.g., rephaser control 50, method 150) in which the slice refocusing gradient is omitted from the gradients applied at 304. As a result of the omitting slice refocusing gradient at 304 of the method 300, background signals are suppressed in the image being reconstructed, resulting in a relative increase in MR signal strength near the catheter coils (e.g., increased signal magnitude) as represented in the reverse polarization components thereof.

The gradients applied at 304 can also include applying respective phase and frequency encoding gradients. The phase-encoding gradient can be applied at 304 along a perpendicular axis (e.g., Y-axis) to introduce spatially dependent phase shifts, encoding positional information along that axis. The encoding gradients can be applied at 304 further can include applying a frequency-encoding gradient along the third axis (e.g., X-axis) during signal acquisition to create a linear variation in resonance frequency along this axis, enabling spatial encoding of the signal.

At 306, the method includes acquiring the MR signals within a spatial region (e.g., field of view) containing the catheter. For example, the signal acquired at 306 can be 1D MR signal projection (e.g., along a given dimension) that is acquired while the frequency- encoding gradient (at 304) is active and electrical current pulses are being applied to the catheter coil(s) (e.g., responsive to the control at 302). Alternatively, a one-dimensional MR projection can be determined by summing a 2D acquired MR image along one dimension or by summing a 3D acquired MR image along two dimensions). The MR signals (e.g., acquired or determined along a respective one or more dimensions) can be stored in memory as MR data for further processing as described herein.

At 308, an expected MR signal is determined. The expected MR signal can be determined along the same one or more dimensions as the MR signal acquired at 306. For example, the expected MR field that is generated for the active catheter can be computed based on catheter and coil geometry of the physical catheter configuration. The calculation can involve the geometry of the catheter coils, their current position (e.g., determined by one or more localization methods described herein) and orientation, as well as the electrical current in the coils. In some examples, the catheter and coil behavior can be defined in one or more mathematical models, which can be configured according to the Biot-Savart law. The computed expected MR signal for the catheter further can be defined as a 1D projection of the expected magnetic field that is determined at 308. As a further example, the 1D MR projection acquired/determined at 306 can represent the B_{z} component of the acquired MR field (e.g., the component along the direction of the main magnetic field of the MRI scanner). Additionally, the expected (e.g., simulated) magnetic field likewise can be computed as a 1D projection along the B_{z} component. The expected MR field can be stored in memory as a vector or set of vectors for different possible locations describing the expected MR field.

At 310, the method includes matching the acquired MR signal(s) (acquired at 306) and the expected MR signal(s) (determined at 308). For example, the matching can be based on a dot product of the expected MR signal and 1D acquired data that is computed for each possible offset of the expected MR signal to the 1D data to find the point where the value of the dot product is maximal. In one example, the expected MR signal can be translated in space to define an expected MR signal for every possible catheter location or a subset of possible locations. The catheter location (e.g., based on a respective translations of the expected MR signal) corresponding to the maximum dot product between expected signal and acquired MR signal is defined as the actual catheter location. In another example, the acquired 1D data representing the acquired MR signal, can be translated throughout space and the expected signal can represent the expected signal for a respective fixed location, and the spatial translation resulting in the maximum dot product can be used to determine the catheter location. While the above example of pattern matching has been described as being implemented with respect to 1D projections, which can be executed more efficiently (e.g., faster) using the dot product function, the matching is not limited to 1D computations. For example, the acquired MR signals and corresponding expected MR signals can be determined in 2D or 3D image space and the matching at 310 can be implemented in 2D or 3D spatial domains. In some examples, it is possible to acquire more than 3 projections of the MR signal and the matching at 310 can perform a least squares optimization (or other metric) to find the spatial coordinates of the catheter.

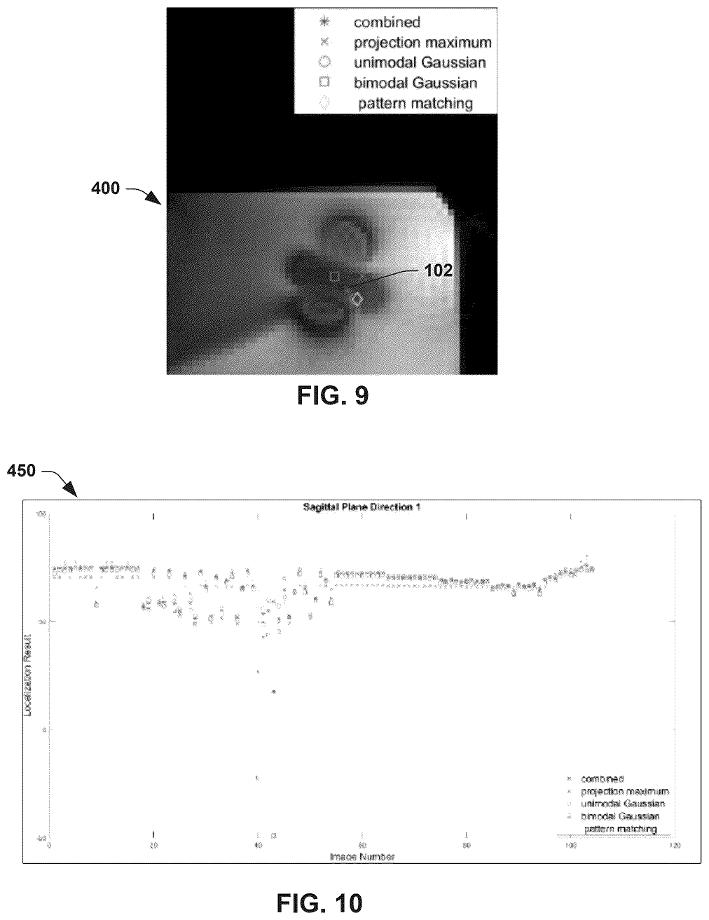

FIGS. 9 and 10 are examples of location data that can be determined according to the approach(es) described herein (sec, e.g., FIGS. 1-8). FIG. 9 is an example image 400 showing results of a localized catheter device (e.g., coordinates of device 18) based on localization data computed by the localization method (e.g., localization method 40) according to different analysis and reconstruction methods (e.g., reconstruction and analysis methods 44, 50, 52, and 54). A combined location is also shown at 102, such as combined from other locations determined from localization data produced by other instances of the localization method based on various reconstruction and analysis methods and a number of respective image measurements.

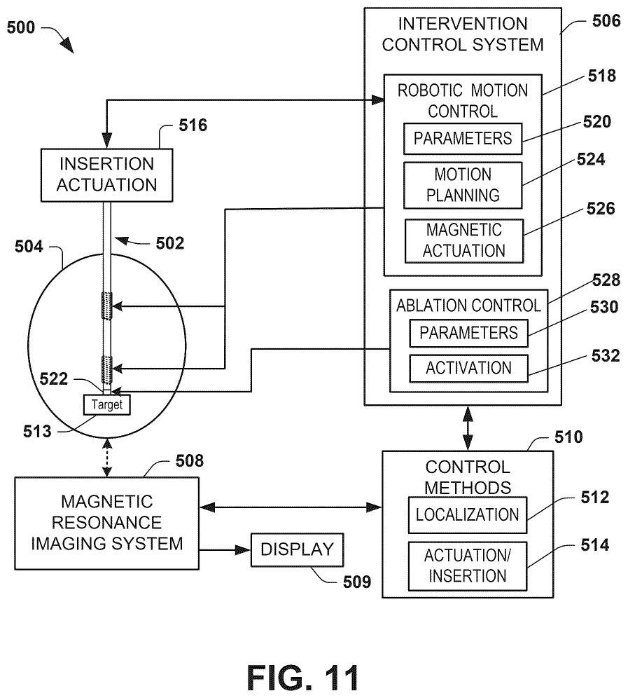

FIG. 10 shows a plot 450 of localization data in a single dimension (e.g., of multi-dimensional space) for different analysis and reconstruction methods, which can be used to analyze each reconstruction and determine coil location for the catheter. The results can be across reconstructions and analysis methods, such as to provide location coordinate results, such as shown in FIG. 11.

As a further example, FIG. 11 depicts an example of an interventional system 500 representative of delivery of ablation therapy via a catheter 502 that is insertable percutaneously into a patient's body, demonstrated schematically at 504. While the example system 500 of FIG. 11 is described with respect to delivery of ablation therapy by the catheter 502, the catheter can be configured to control delivery of other types of therapy or control signal measurements of electrical or environmental conditions based on the location of the catheter, such as described herein. The system 500 includes an intervention control system 506 that is configured to implement one or more interventional control functions to operate one or more devices for diagnosing and/or treating the patient's body 504. The intervention control system 506 is a useful example of the control system 28 shown in FIG. 1. The system 500 also includes an imaging system demonstrated in this example as a magnetic resonance imaging (MRI) system (also referred to as an imaging system) 508; although, other imaging modalities can be used in other examples.

The system 500 also can include one or more control methods 510, which can be implemented according to control methods described herein (e.g., the control methods 36 of FIG. 1). As a further example, the control methods 510 can include a catheter localization method 512 to determine a location (e.g., coordinates of spatial position) as well as an orientation of the catheter, such as described herein (see, e.g., FIGS. 1-10). Thus, the description of FIG. 11, particularly the localization method 512, can refer to certain aspects of FIGS. 1-10.

Additionally, or alternatively, the control methods 510 can include an actuation and insertion control method 514, such as described herein (e.g., control methods 60). Accordingly, the description of FIG. 3 also refers to certain aspects of FIG. 1. As another additional or alternative example, the catheter 502 can be implemented as a hybrid magnetically actuated flexible catheter that includes variable length insertion stiffening clement adapted for insertion within a lumen of the catheter, as also described herein.

For example, the control methods 510 can be configured to provide control inputs to the imaging system 508 and the intervention control system 506. The control methods 510 can be implemented separately from or as part of one or both of the imaging system 508 and/or intervention control system 506. The control methods 510 can be implemented as machine instructions (executable by one or more processors) to generate one or more control inputs in response to imaging data acquired by the imaging system 508. The control methods 510 can implement the localization 512 and actuation/insertion control 514 (e.g., corresponding to method 40 or 60) for the imaging system 508 and the intervention control system 506.

The imaging system 508 can acquire image data of the patient's body including the catheter 502 and the corresponding target site 513. For example, the imaging system 508 (e.g., an MRI scanner) is configured to image the catheter tip (of device 18) and the target tissue (e.g., tissue 20), such as on the pulmonary vein or other tissue structure and provide corresponding image data in substantially real-time (e.g., generate a new set of image data about every 20 ms to 50 ms). The image data can be raw MR data that is preprocessed by the imaging system 508 and utilized by the control methods 510 to determine location (e.g., spatial coordinates) of the catheter 502 and provide corresponding input control parameters to the MRI system 508 for active sensing of the target 513 and catheter device 502. Additionally, the control methods 510 can provide corresponding input control parameters to the intervention control system 506 for controlling intervention with respect to the patient's body 504. The imaging system 508 can further generate an output visualization to provide a real-time display 509 according to the image being acquired.

In the following example, the intervention is provided via the catheter 502 that is inserted percutaneously, which can be implemented based on location data computed by the localization method 40 (e.g., real-time or near real-time location data). However, it is understood that the interventional system 500 can implement a variety of other diagnostic and treatment devices, which may be inserted internally within the body 504 or be provided externally (e.g., sources of radiation) applied to the internal target 513 that is actively localized.

In the example of FIG. 3, the intervention control system 506 includes a robotic motion control 518 to control operation of an arrangement of motion components (e.g., motors, actuators, coils or the like) to position the catheter 502 at a desired position relative to the patient's body. For example, the robotic motion control 518 can include a motion planning module 524 to determine control parameters 520 for adjusting the position and/or orientation of the catheter 502 in a coordinate system of the patient and imaging system 508. The robotic motion control 518 can be fully automated in response to the control parameters 520 provided by motion planning 524 and the control methods 510. In other examples, the robotic motion control can be co-robotically controlled, meaning that the parameters 520 can be modified based on user interaction and resulting catheter positioning is robotically assisted.

In the example of FIG. 3, the system 500 includes an axial actuation component 516 that can be utilized to adjust the axial position of a distal end of the catheter 502 in response to the control signals provided by robotic motion control 518. For example, the motion planning module 524 can determine control parameters 520 for axially adjusting the position and/or orientation of the catheter 502 in a coordinate system of the patient and imaging system 508. The axial actuation 516 can include a linear actuator (e.g., linear motor or servo motor) configured to advance the catheter body generally in an axial direction along a trajectory to position the catheter tip 522 (or another distal portion thereof) into contact with the target 513.

In addition to adjusting the position of the catheter axially along the path via the actuation 516, the intervention control system 506 can include magnetic actuation control 526 to provide for translational motion with respect to three orthogonal axes in three-dimensional space. For example, the catheter magnetic actuation control 526 controls current applied to coils attached to the catheter body to affect deflection of the catheter in two orthogonal axes relative to its longitudinal axis. For example, the catheter body can include a polymer tubing that is embedded with multiple sets of corresponding current carrying coils.

The catheter 502 can be steered in a couple of different manners. As one example, the magnetic field of the MRI system 508 can be used to directly actuate the catheter. Alternatively, or additionally, a small robot (e.g., placed near the patient's knees) can move the catheter 502 axially, rotate, and bend. The catheter 502 itself would have an arrangement of coils embedded to help identify its location and orientation from the image data.

As a further example, the catheter 502 can be implemented as a MR-actuated steerable catheter that can cause deflection along the catheter body along transverse to the longitudinal axis thereof in response to current supplied by the robotic motion control 518. For example, current through the coils generates magnetic torque when subjected to the magnetic field provided by the MRI system 508. The currents can be controlled remotely via the magnetic actuation control 526 automatically or in response to a user input to steer the catheter in conjunction with the axial actuation 516 to move the distal end 522 of the catheter along a given trajectory, such as to contact the target site. The catheter motion can be achieved, for example, by interlacing the imaging and catheter control signals in the MRI system.

As the moment applied by the actuation 516 is dependent on the relative orientation of the actuator coils and the magnetic field vector of the MRI system 508, the external loading on the catheter changes with deflection. Therefore, the robotic motion control 518 is configured to calculate forward kinematics (catheter deflection for a given set of actuator currents) requires the solution of a system of nonlinear equations. The robotic motion control 518 also computes corresponding inverse kinematics because of the complexity of the kinematics. In the given formulation, it would be possible to derive analytic expressions for the Jacobian. That is, Jacobian-based methods can be effectively used for inverse kinematics calculations. For example, the robotic motion control 518 of intervention control system 506 can employ the damped least squares method, which avoids stability problems in the neighborhoods of singularities.

In an example, where the rigid shaft is used (e.g., for axial movement within or over the catheter body), the proximal part of the catheter has a fixed shape due to the rigid shaft, and as a result would not bend. The forward kinematics, the inverse kinematics and the Jacobian are calculated by taking the pose of the rigid shaft into account with respect to the length of the catheter body. The control algorithm is further configured to determine how the rigid insert needs to be inserted along with the regular catheter insertion and the coil actuation, based on the desired motion of the flexible portion of the catheter.

As described herein, the location of the catheter tip 522 and the target tissue 513 are simultaneously measured by the intra-operative MRI system 508 in real-time to implement the localization and control methods 512 and 514. For instance, the control methods 510 can implement an active sensing scheme to control the intra-operative imaging system 508, optimally selecting position and orientation of the image slices to be acquired as well as other imaging parameters, for desired localization of the target and the catheter. The motion planning 524 and actuation control 526 of the robotic motion control 518 can utilize the target and catheter location information computed by localization methods 40 based on the acquired MR data and reconstructed image data from the intra-operative MRI system as well as active control inputs selected for each time step to actively control the position and/or shape catheter tip to track the target tissue.

Once the distal end 522 of the catheter 502, which can include an ablation delivery structure (e.g., one or more electrodes or other structures adapted for ablation delivery), is in contact with the target site 513, the ablation control 528 can deliver ablated therapy to the target. The ablation control 528 can operate based upon a set of ablation parameters 530 and an activation function 532 to supply the ablative therapy via the delivery mechanism (e.g., at the distal end 522) according to the parameters 530. In one example, the ablation control 528 can supply RF energy to the electrode at the distal end (e.g., at the tip or a distal portion) 522 of the catheter 502 and activate the RF energy to ablate the tissue at the contact site of the target 513 in response to the parameters 530 specifying amplitude, frequency, duration (e.g., duty cycle) of energy. In other examples, the ablation delivery structure can be configured to deliver one or more other forms of ablation, such as cryoablation, laser ablation, chemical ablation, etc. In an example, the catheter is an ablation catheter adapted to perform brain/cerebral catheter ablations, which can be implemented during interventional MRI of the brain. During application of the therapy or other intervention, the intervention control system 506, including robotic motion control 518 and insertion actuation 514, further can adjust the position of the distal end of the catheter across the target 513 to achieve a sufficient amount of ablation on the target site. For example, the distal end of the catheter can be moved in a sequence of patterns and ablate the tissue at each of a plurality of locations across the surface or through the volume of the target tissue. The sequence of patterns can be automated or responsive to user controls.

In order to achieve successful ablation, the catheter tip 522 needs to remain in constant contact with the tissue when the activation function 532 applies ablative therapy. Such contact should be maintained and follow the trajectory specified by the operator, despite disturbances from blood flow and tissue motions, as mentioned above. Accordingly, in some examples, the robotic motion control 518 dynamically corrects the position of the catheter to compensate for (e.g., cancel) the cardiac motions and/or perturbations resulting from blood flow, and/or other physiological effects. For instance, the robotic motion control 518 can employ active relative motion cancellation methods. The motion control 518 actively tracks the motion of the deformable tissue, so as to dynamically adjust the position of the tip of the catheter to remain stationary relative to the tissue. Once the tip of the catheter is “stationary,” the motion planning methods 524 can be performed with respect to a stationary tissue frame, and the resulting catheter motion can be added to the active tracking motion.

To reduce the computational complexity associated with such active tracking, the motion control can take advantage of the quasi-periodic nature of the target tissue motion (e.g., wall motion synchronized with the patient's heartbeat), and the perturbations on the catheter motion due to non-steady blood flow. The motion control can employ generalized adaptive filters for estimating the upcoming motion of the target tissue and the catheter. Once estimates of the upcoming catheter shape and target motions are available, even if they are not within expected operating parameters, these estimates can be used to reduce the uncertainties in system dynamics. Reduced uncertainty facilitates the use of unimodal distributions in representing uncertainties in the models being implemented and can even enable use of linearized models in some examples.

### EXAMPLE EMBODIMENTS

Several aspects of the present technology are set forth in the following numbered examples.

Example 1. A system, comprising:
one or more non-transitory media storing data and executable instructions; and
a processor configured to access the non-transitory media and execute instructions comprising:
   image reconstruction code programmed to reconstruct images based on acquired magnetic resonance (MR) data and provide reconstructed image data, in which at least some of the MR data includes a representation of a device within a field of view;
   localization code programmed to provide localization data representative of at least one of a location, orientation and/or shape of the device based on the reconstructed image data; and
   off-resonance control code programmed to control an MR imaging system to provide radio frequency (RF) pulses at one or more off-resonant frequencies to excite off- resonance spins near a current-carrying coil carried by the device within the field of view, such that the acquired MR data is representative of the off-resonance excitation during MR image acquisition.

Example 2. The system of example 1, further comprising the MR imaging system, in which the MR imaging system is configured to generate the acquired MR data based on RF pulses and magnetic field gradients provided within the field of view during the MR image acquisition.

Example 3. The system of example 2, wherein the off-resonance control code is programmed to control at least some of the RF pulses provided by the MR imaging system concurrently with current pulses provided to the current carrying coil during the MR image acquisition.

Example 4. The system of example 3, wherein the off-resonance control code is programmed to control the at least some of the RF pulses to be provided at a frequency that is offset from the Larmor frequency to selectively excite off-resonant spins adjacent to the current-carrying coil.

Example 5. The system according to any one of the preceding examples, wherein the instructions further comprise:
rephaser control code programmed to control the MR imaging system to omit RF refocusing pulses during the MR image acquisition, such that the acquired MR data is representative of MR signals in the field of view in the absence of RF refocusing pulses during the MR image acquisition.

Example 6. The system according to example 5, further comprising an acquisition user interface programmed to select at least one of the off-resonance control code and the rephaser control code to be implemented by the imaging system during the MR image acquisition, the acquired MR data being provided based on the selected off-resonance control code and/or rephaser control code.

Example 7. The system according to any one of the preceding examples, wherein the instructions further comprise:
reverse polarization code programmed to control the image reconstruction code to apply reverse polarization reconstruction with respect to the acquired MR data and provide the reconstructed image data.

Example 8. The system according to any one of the preceding examples, wherein the instructions further comprise:
pattern matching code programmed to provide the localization data based on the acquired MR data and an expected MR signal.

Example 9. The system according to example 8, further comprising a reconstruction user interface programmed to invoke at least one of the reverse polarization code and the pattern matching code.

Example 10. The system according to any one of examples 8 or 9, wherein the pattern matching code is programmed to compare the acquired MR data to expected MR signal generated based on Biot-Savart simulations for the device.

Example 11. The system according to any one of examples 2, 3, or 4, wherein the localization code is further programmed to:
control the MR imaging system to generate multiple sets of the acquired MR data based on different parameters for the off-resonance control code and/or the rephaser control code;
control the image reconstruction code to provide a respective set of the reconstructed image data based on each of the sets of the acquired MR data;
compute respective localization data for the device based on each set of the reconstructed image data; and
combine the respective localization data to determine the spatial location of the device.

Example 12. The system according to any one of examples 1, 2, or 3, wherein the reverse polarization code and/or pattern matching code are implemented as part of the image reconstruction code or are applied to one or more reconstructed images after image reconstruction and/or processing thereof.

Example 13. The system according to any one of the preceding examples, wherein the localization code is programmed to control the image reconstruction code to produce a plurality of reconstructed image sets based on different sets of the acquired MR data, conjugates of reconstructed images, different weight matrices, and/or different coil sensitivity maps.

Example 14. The system according to any one of the preceding examples, wherein the system comprises the device and the device is a catheter, the catheter includes a plurality of multi-axial coils disposed about a body of the catheter, and each of the coils has a respective axis positioned to provide for selective movement of the catheter relative to the respective axis.

Example 15. The system of example 14,
wherein the instructions include actuation control configured to provide electrical current to at least one actuation coil at a distal end portion of the catheter to deflect the distal end portion of the catheter based on the localization data, and/or
the system further comprises an insertion actuator being configured to move at least the distal end portion of the catheter axially in response to an actuation control signal, in which the instructions include insertion control code programmed to provide the actuation control signal based on the localization data.

Example 16. A method, comprising:
controlling electrical current to at least one actuation coil disposed about an elongate flexible body of a device within a field of view of magnetic resonance (MR) imaging system;
controlling the MR imaging system to provide radio frequency (RF) pulses at one or more off-resonant frequencies to excite off-resonance spins near the at least one actuation coil within the field of view, such that the acquired MR data is representative of the off-resonance excitation during MR image acquisition;
providing MR data based on MR signals acquired by the MR imaging system, in which at least some of the MR data includes the MR signals with the device within the field of view;
reconstructing images based on the MR data to provide reconstructed image data; and
providing localization data representative of at least one of a location, orientation and/or shape of the device based on the reconstructed image data.

Example 17. The method of example 16, wherein controlling the MR imaging system further comprises:
controlling application of spatial encoding gradients within the field of view during the MR image acquisition, and
controlling at least some of the RF pulses provided by the MR imaging system concurrently with the electrical current provided to the at least one actuation coil during the MR image acquisition.

Example 18. The method of example 17, wherein the at least some of the RF pulses are provided at a frequency that is offset from the Larmor frequency to selectively excite off-resonant spins adjacent to the at least one actuation coil.

Example 19. The method according to any one of examples 16, 17, or 18, further comprising:
controlling the MR imaging system to omit RF refocusing pulses during the MR image acquisition, such that the acquired MR data is representative of MR signals in the field of view in the absence of RF refocusing pulses during the MR image acquisition.

Example 20. The method according to example 19, wherein, responsive to a user input instruction, the method comprises selectively controlling at least one of:
providing the RF pulses at one or more off-resonant frequencies; and
omitting the RF refocusing pulses, such that the acquired MR data is based on the selective controlling.

Example 21. The method according to any one of examples 16, 17, 18, or 19, wherein reconstructing images further comprises:
applying reverse polarization reconstruction with respect to the acquired MR data to provide the reconstructed image data.

Example 22. The method according to any one of examples 16, 17, 18, 19, or 21, wherein providing localization data comprises matching based on the acquired MR data and an expected MR signal.

Example 23. The method according to example 22, wherein the matching compares the acquired MR data to the expected MR signal generated based on Biot-Savart simulations for the device.

Example 24. The method according to example 20, further comprising:
controlling the MR imaging system to generate multiple sets of the acquired MR data based on different parameters for providing the RF pulses at one or more off- resonant frequencies and/or omitting the RF refocusing pulses;
providing a respective set of the reconstructed image data based on each of the sets of the acquired MR data;
computing respective localization data for the device based on each set of the reconstructed image data; and
combining the respective localization data to determine the spatial location of the device.

Example 25. The method according to example 21, wherein applying reverse polarization reconstruction further comprises:
producing a plurality of reconstructed image sets based on different sets of the acquired MR data, conjugates of reconstructed images, different weight matrices, and/or different coil sensitivity maps.

Example 26. The method according to any one of examples 16, 17, 18, 19, 20, 21, 22, 23, or 24, wherein the device is a catheter, the at least one actuation coil includes a plurality of multi-axial actuation coils disposed about a distal body portion of the catheter, and each of the coils has a respective axis positioned to provide for selective movement of the catheter relative to the respective axis.

Example 27. The method according to example 26,
providing at least one actuation control signal to provide electrical current to at least one of the actuation coils at the distal body portion of the catheter to deflect the distal body portion of the catheter based on the localization data, and/or
providing an insertion control signal to move at least the distal body portion of the catheter axially based on the localization data.

Example 28. A non-transitory machine-readable medium to store executable instructions, the instructions to cause a processor to perform a method according to any one of examples 16-25 or 27.

Example 29. A method, comprising:
controlling electrical current to at least one actuation coil disposed about an elongate flexible body of a device within a field of view of magnetic resonance (MR) imaging system;
controlling the MR imaging system to omit RF refocusing pulses during the MR image acquisition, such that the acquired MR data is representative of MR signals in the field of view in the absence of RF refocusing pulses during the MR image acquisition;
providing MR data based on MR signals acquired by the MR imaging system, in which at least some of the MR data includes the MR signals with the device within the field of view;
reconstructing images based on the MR data to provide reconstructed image data; and
providing localization data representative of at least one of a location, orientation and/or shape of the device based on the reconstructed image data.

Example 30. A method, comprising:
controlling electrical current to at least one actuation coil disposed about an elongate flexible body of a device within a field of view of magnetic resonance (MR) imaging system;
providing MR data based on MR signals acquired by the MR imaging system, in which at least some of the MR data includes the MR signals with the device within the field of view;
reconstructing images based on the MR data to provide reconstructed image data, in which the reconstructing images includes applying reverse polarization reconstruction with respect to the acquired MR data to provide the reconstructed image data; and
providing localization data representative of at least one of a location, orientation and/or shape of the device based on the reconstructed image data.

Example 31. A method, comprising:
controlling electrical current to at least one actuation coil disposed about an elongate flexible body of a device within a field of view of magnetic resonance (MR) imaging system;
acquiring MR data based on MR signals acquired by the MR imaging system, in which at least some of the MR data includes the MR signals with the device within the field of view; and
providing localization data representative of at least one of a location, orientation and/or shape of the device based on matching the acquired MR data and an expected MR signal.

Example 32. The method according to any one of examples 29, 30, or 31, wherein the device is a catheter, the at least one actuation coil includes a plurality of multi-axial actuation coils disposed about a distal body portion of the catheter, and each of the coils has a respective axis positioned to provide for selective movement of the catheter relative to the respective axis.

Example 33. A non-transitory machine-readable medium to store executable instructions, the instructions to cause a processor to perform a method according to any one of examples 29, 30, or 31.

In view of the foregoing structural and functional description, those skilled in the art will appreciate that portions of the systems and methods described herein may be embodied as a method, data processing system, or computer program product. Accordingly, these portions of such systems and methods may take the form of an entirely hardware embodiment, an entirely software embodiment, or an embodiment combining software and hardware. Furthermore, portions of the systems and methods may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., as part of the imaging system), or other programmable data processing apparatus (or a combination of devices and circuits) to produce a machine, such that the instructions, which execute via the processor, implement the functions specified in the block or blocks.

These computer-executable instructions may also be stored in computer- readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

All references, publications, and patents cited in the present application are herein incorporated by reference in their entirety.

What is claimed is:

1. A system, comprising:
one or more non-transitory media storing data and executable instructions; and
a processor configured to access the non-transitory media and execute the instructions, the instructions comprising:
image reconstruction code programmed to reconstruct images based on acquired magnetic resonance (MR) data and provide reconstructed image data, in which at least some of the MR data includes a representation of a device within a field of view;
localization code programmed to provide localization data representative of at least one of a location, orientation and/or shape of the device based on the reconstructed image data; and
off-resonance control code programmed to control an MR imaging system to provide radio frequency (RF) pulses at one or more off-resonant frequencies to excite off- resonance spins near a current-carrying coil carried by the device within the field of view, such that the acquired MR data is representative of the off-resonance excitation during MR image acquisition.

2. The system of claim 1, further comprising the MR imaging system, in which the MR imaging system is configured to generate the acquired MR data based on RF pulses and magnetic field gradients provided within the field of view during the MR image acquisition.

3. The system of claim 2, wherein the off-resonance control code is programmed to control at least some of the RF pulses provided by the MR imaging system concurrently with current pulses provided to the current carrying coil during the MR image acquisition.

4. The system of claim 3, wherein the off-resonance control code is programmed to control the at least some of the RF pulses to be provided at a frequency that is offset from the Larmor frequency to selectively excite off-resonant spins adjacent to the current-carrying coil.

5. The system according to claim 1, wherein the instructions further comprise:
rephaser control code programmed to control the MR imaging system to omit RF refocusing pulses during the MR image acquisition, such that the acquired MR data is representative of MR signals in the field of view in the absence of RF refocusing pulses during the MR image acquisition.

6. The system according to claim 5, further comprising an acquisition user interface programmed to select at least one of the off-resonance control code and the rephaser control code to be implemented by the imaging system during the MR image acquisition, the acquired MR data being provided based on the selected off-resonance control code and/or rephaser control code.

7. The system according to claim 1, wherein the instructions further comprise:
reverse polarization code programmed to control the image reconstruction code to apply reverse polarization reconstruction with respect to the acquired MR data and provide the reconstructed image data.

8. The system according to claim 1, wherein the instructions further comprise:
pattern matching code programmed to provide the localization data based on the acquired MR data and an expected MR signal.

9. The system according to claim 8, wherein the instructions further comprise:
reverse polarization code programmed to control the image reconstruction code to apply reverse polarization reconstruction with respect to the acquired MR data and provide the reconstructed image data; and
a reconstruction user interface programmed to invoke at least one of the reverse polarization code and the pattern matching code.

10. The system according to claim 8, wherein the pattern matching code is programmed to compare the acquired MR data to expected MR signal generated based on Biot-Savart simulations for the device.

11. The system according to claim 1, wherein the localization code is further programmed to:
control the MR imaging system to generate multiple sets of the acquired MR data based on different parameters for the off-resonance control code and/or the rephaser control code;
control the image reconstruction code to provide a respective set of the reconstructed image data based on each of the sets of the acquired MR data;
compute respective localization data for the device based on each set of the reconstructed image data; and
combine the respective localization data to determine the spatial location of the device.

12. The system according to claim 1, wherein the localization code is programmed to control the image reconstruction code to produce a plurality of reconstructed image sets based on different sets of the acquired MR data, conjugates of reconstructed images, different weight matrices, and/or different coil sensitivity maps.

13. The system according to claim 1, wherein the system comprises the device and the device is a catheter, the catheter includes a plurality of multi-axial coils disposed about a body of the catheter, and each of the coils has a respective axis positioned to provide for selective movement of the catheter relative to the respective axis.

14. The system of claim 13,
wherein the instructions include actuation control configured to provide electrical current to at least one actuation coil at a distal end portion of the catheter to deflect the distal end portion of the catheter based on the localization data, and/or
wherein the system further comprises an insertion actuator being configured to move at least the distal end portion of the catheter axially in response to an actuation control signal, in which the instructions include insertion control code programmed to provide the actuation control signal based on the localization data.

15. A method, comprising:
controlling electrical current to at least one actuation coil disposed about an elongate flexible body of a device within a field of view of magnetic resonance (MR) imaging system;
controlling the MR imaging system to provide radio frequency (RF) pulses at one or more off-resonant frequencies to excite off-resonance spins near the at least one actuation coil within the field of view, such that the acquired MR data is representative of the off- resonance excitation during MR image acquisition;
providing MR data based on MR signals acquired by the MR imaging system, in which at least some of the MR data includes the MR signals with the device within the field of view;
reconstructing images based on the MR data to provide reconstructed image data; and
providing localization data representative of at least one of a location, orientation and/or shape of the device based on the reconstructed image data.

16. The method of claim 15, wherein controlling the MR imaging system further comprises:
controlling application of spatial encoding gradients within the field of view during the MR image acquisition, and
controlling at least some of the RF pulses provided by the MR imaging system concurrently with the electrical current provided to the at least one actuation coil during the MR image acquisition.

17. The method of claim 16, wherein the at least some of the RF pulses are provided at a frequency that is offset from the Larmor frequency to selectively excite off-resonant spins adjacent to the at least one actuation coil.

18. The method according to claim 15, further comprising:
controlling the MR imaging system to omit RF refocusing pulses during the MR image acquisition, such that the acquired MR data is representative of MR signals in the field of view in the absence of RF refocusing pulses during the MR image acquisition.

19. The method according to claim 18, wherein, responsive to a user input instruction, the method comprises selectively controlling at least one of:
providing the RF pulses at one or more off-resonant frequencies; and
omitting the RF refocusing pulses, such that the acquired MR data is based on the selective controlling.

20. The method according to claim 15, wherein reconstructing images further comprises:
applying reverse polarization reconstruction with respect to the acquired MR data to provide the reconstructed image data.

21. The method according to claim 20, wherein applying reverse polarization reconstruction further comprises:
producing a plurality of reconstructed image sets based on different sets of the acquired MR data, conjugates of reconstructed images, different weight matrices, and/or different coil sensitivity maps.

22. The method according to claim 15, wherein providing localization data comprises matching based on the acquired MR data and an expected MR signal.

23. The method according to claim 22, wherein the matching compares the acquired MR data to the expected MR signal generated based on Biot-Savart simulations for the device.

24. The method according to claim 15, wherein the device is a catheter, the at least one actuation coil includes a plurality of multi-axial actuation coils disposed about a distal body portion of the catheter, and each of the coils has a respective axis positioned to provide for selective movement of the catheter relative to the respective axis.

25. The method according to claim 24,
controlling at least one actuation control signal to provide electrical current to at least one of the actuation coils at the distal body portion of the catheter to deflect the distal body portion of the catheter based on the localization data, and/or
controlling an insertion control signal to move at least the distal body portion of the catheter axially based on the localization data.

26. A non-transitory machine-readable medium to store executable instructions, the instructions to cause a processor to perform the method according to claim 15.
